(19) United States
(12) Patent Application Publication
(10) Pub. No.: US 2025/0349400 A1
(43) Pub. Date: Nov. 13, 2025

(54) DEVICE AND METHOD TO CREATE A LOW-POWERED APPROXIMATION OF COMPLETED SETS OF DATA

(71) Applicant: HAPPY HEALTH, INC., Austin, TX (US)

(72) Inventors: Conrad W. ROSENBROCK, Austin, TX (US); David E. CLIFT-REAVES, Austin, TX (US); Arnulf GRAF, Austin, TX (US); Derek HORKEL, Austin, TX (US); David CARR, Austin, TX (US)

(73) Assignee: HAPPY HEALTH, INC., Austin, TX (US)

(21) Appl. No.: 18/871,247

(22) PCT Filed: Jun. 2, 2023
(86) PCT No.: PCT/US2023/024292

§ 371 Date: Dec. 3, 2024

Related U.S. Application Data
(60) Provisional application No. 63/348,796, filed on Jun. 3, 2022.

(51) Int. Cl. G16H 10/60; A61B 5/00; A61B 5/0205; A61B 5/024; A61B 5/053

(52) U.S. Cl. CPC G16H 10/60; A61B 5/002; A61B 5/0205; A61B 5/053; A61B 5/6801; A61B 5/02416; A61B 5/02438; A61B 2562/0219; A61B 2562/06

(57) ABSTRACT

A method of creating a low-powered approximation of one or more completed sets of data is provided. A lower resolution is sampled, at a predetermined interval distribution, of one or more of sensors: accelerometer, electrodermal activity sensor, photoplethysmographic (PPG) sensor; impedance sensor, gyroscopic sensor, and/or a radio sensor. A combined uncertainty from the sensors is determined. Error change in a predicting a signal is estimated. The predetermined interval distribution is modified based upon the combined uncertainty and using phase-locked loops at one or more targeted frequencies, which are adjusted in real-time. The sampling of the sensors is modified, in real time based on the estimated error change, to be one of random, sparse, and/or high resolution.

### CROSS-REFERENCE TO RELATED APPLICATION

This application claims priority to U.S. Provisional Patent Application No. 63/348,796, filed in the U.S. Patent and Trademark Office on Jun. 3, 2022, all of which is incorporated herein by reference in its entirety for all purposes.

### FIELD

The present disclosure relates generally to small form factor wearable devices with power limitations that use sensors to approximate sets of data such as heart rate, heart rate variability, oxygen saturation (SpO2), hydration, blood pressure, blood glucose, electrodermal activity, and/or respiratory rate.

### BACKGROUND

Wearable devices come in different form factors. Some wearables require a tether, but increasingly the wearable is self-contained and battery powered. The wearables include one or more sensor and/or sensor modules that are configured to contact part of a user's or wearer's body. These sensors are used to calculate or measure characteristics that are then used for determining biometrics of interest, for example heart rate.

### BRIEF DESCRIPTION OF THE DRAWINGS

Details of one or more aspects of the subject matter described in this disclosure are set forth in the accompanying drawings and the description below. However, the accompanying drawings illustrate only some typical aspects of this disclosure and are therefore not to be considered limiting of its scope. Other features, aspects, and advantages will become apparent from the description, the drawings and the claims.

FIG. 1 shows an example of a system such as a wearable device for implementing certain aspects of the present technology;

FIG. 2 is a flowchart illustrating a method for assisting a user in attaining a wellness goal in accordance with the present disclosure;

FIG. 3A illustrates a diagram showing creation of random samples to compute uncertainty from a single sensor;

FIG. 3B illustrates a graph showing frequency detection vs. compression;

FIG. 4 illustrates a graph showing probability of selection vs. sampling interval distribution; and

FIG. 5 illustrates a process for adjusting sample intervals using uncertainty matrix eigenvalues and signal-to-noise ratio (SNR) limits of sensors.

### DETAILED DESCRIPTION

As used herein, the terms “comprises,” “comprising,” “includes,” “including,” “has,” “having” or any other variation thereof, are intended to cover a non-exclusive inclusion. For example, a process, product, article, or apparatus that comprises a list of elements is not necessarily limited to only those elements but can include other elements not expressly listed or inherent to such process, process, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

The term substantially, as used herein, is defined to be essentially conforming to the particular dimension, shape or other word that substantially modifies, such that the component need not be exact. For example, substantially cylindrical means that the object resembles a cylinder, but can have one or more deviations from a true cylinder.

The term “coupled” is defined as connected, whether directly or indirectly through intervening components, and is not necessarily limited to physical connections. The connection can be such that the objects are permanently connected or releasably connected. The term “comprising” means “including, but not necessarily limited to”; it specifically indicates open-ended inclusion or membership in a so-described combination, group, series and the like.

For many sensor modalities and configurations, the power consumed is proportional to the number of samples taken by the sensor. Reducing the number of samples required to reconstruct a signal therefore increases the wear time of the sensor between charges. Modulating the transmit power of a sensor can also reduce power consumption when it will not adversely affect signal quality.

This disclosure deals broadly with methods to reduce power consumption in sensors used to predict biometrics by reducing the number of samples taken by the sensors, or by modulating the transmit power of the sensors. In a more general sense, it describes methods for minimizing power consumption with respect to desired information content within a signal by altering the distribution of sampling intervals and power modulation for one or more sensors.

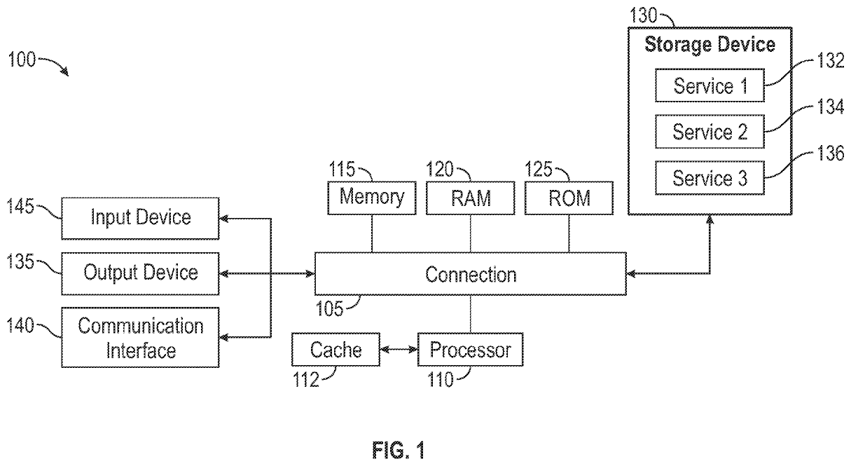

FIG. 1 shows an example of computing system 100, which can be for example a wearable device, or any component thereof in which the components of the system are in communication with each other using connection 105. The computing system 100 can be utilized for any or all of the features and steps in the present disclosure. Connection 105 can be a physical connection via a bus, or a direct connection into processor 110, such as in a chipset architecture. Connection 105 can also be a virtual connection, networked connection, or logical connection.

In some examples, computing system 100 is a distributed system in which the functions described in this disclosure can be distributed within a datacenter, multiple data centers, a peer network, etc. In some examples, one or more of the described system components represents many such components each performing some or all of the function for which the component is described. In some embodiments, the components can be physical or virtual devices.

Example system 100 includes at least one processing unit (CPU or processor) 110 and connection 105 that couples various system components including system memory 115, such as read-only memory (ROM) 120 and random access memory (RAM) 125 to processor 110. Computing system 100 can include a cache of high-speed memory 112 connected directly with, in close proximity to, or integrated as part of processor 110.

Processor 110 can include any general purpose processor and a hardware service or software service, such as services 132, 134, and/or 136 stored in storage device 130, configured to control processor 110 as well as a special-purpose processor where software instructions are incorporated into the actual processor design. Processor 110 may essentially be a completely self-contained computing system, containing multiple cores or processors, a bus, memory controller, cache, etc. A multi-core processor may be symmetric or asymmetric.

To enable user interaction, computing system 100 includes an input device 145, which can represent any number of input mechanisms, such as sensors (for example accelerometer, electrodermal activity sensor, photoplethysmographic (PPG) sensor; impedance sensor, gyroscopic sensor, and/or a radio sensor), a microphone for speech, a touch-sensitive screen for gesture or graphical input, keyboard, mouse, motion input, speech, etc. Computing system 100 can also include output device 135, which can be one or more of a number of output mechanisms known to those of skill in the art. In some instances, multimodal systems can enable a user to provide multiple types of input/output to communicate with computing system 100. Computing system 100 can include communications interface 140, which can generally govern and manage the user input and system output. There is no restriction on operating on any particular hardware arrangement, and therefore the basic features here may easily be substituted for improved hardware or firmware arrangements as they are developed.

Storage device 130 can include a non-volatile memory device and can include a hard disk or other types of computer readable media which can store data that are accessible by a computer, such as magnetic cassettes, flash memory cards, solid state memory devices, digital versatile disks, cartridges, random access memories (RAMs), read-only memory (ROM), and/or some combination of these devices.

The storage device 130 can include software services, servers, services, etc., that when the code that defines such software is executed by the processor 110, it causes the system to perform a function. In some examples, a hardware service that performs a particular function can include the software component stored in a computer-readable medium in connection with the necessary hardware components, such as processor 110, connection 105, output device 135, etc., to carry out the function.

For clarity of explanation, in some instances, the present technology may be presented as including individual functional blocks including functional blocks comprising devices, device components, steps or routines in a method embodied in software, or combinations of hardware and software.

Any of the steps, operations, functions, or processes described herein may be performed or implemented by a combination of hardware and software services or services, alone or in combination with other devices. In some examples, a service can be software that resides in memory of a client device and/or one or more servers of a content management system and perform one or more functions when a processor executes the software associated with the service. In some embodiments, a service is a program or a collection of programs that carry out a specific function. In some examples, a service can be considered a server. The memory can be a non-transitory computer-readable medium.

In some examples, the computer-readable storage devices, mediums, and memories can include a cable or wireless signal containing a bit stream and the like. However, when mentioned, non-transitory computer-readable storage media expressly exclude media such as energy, carrier signals, electromagnetic waves, and signals per se.

Methods according to the above-described examples can be implemented using computer-executable instructions that are stored or otherwise available from computer-readable media. Such instructions can comprise, for example, instructions and data which cause or otherwise configure a general purpose computer, special purpose computer, or special purpose processing device to perform a certain function or group of functions. Portions of computer resources used can be accessible over a network. The executable computer instructions may be, for example, binaries, intermediate format instructions such as assembly language, firmware, or source code. Examples of computer-readable media that may be used to store instructions, information used, and/or information created during methods according to described examples include magnetic or optical disks, solid-state memory devices, flash memory, USB devices provided with non-volatile memory, networked storage devices, and so on.

Devices implementing methods according to these disclosures can comprise hardware, firmware and/or software, and can take any of a variety of form factors. Typical examples of such form factors include servers, laptops, smartphones, small form factor personal computers, personal digital assistants, and so on. The functionality described herein also can be embodied in peripherals or add-in cards. Such functionality can also be implemented on a circuit board among different chips or different processes executing in a single device, by way of further example.

The instructions, media for conveying such instructions, computing resources for executing them, and other structures for supporting such computing resources are means for providing the functions described in these disclosures.

The present disclosure addresses power consumption in the wearable devices 100 by using custom distributions of sampling intervals and transmit power levels for sensors 145, for example randomly subsampled sensors 145 in a low power, small form factor wearable. The disclosure discusses methods for modifying the time intervals between samples taken. However, the same methodologies can be identically applied to transmit power of the sensors 145, for example, increasing the time between samples reduces the total number of samples and the power achieved. A power reduction can also be achieved by reducing the transmit power in the sensor and keeping the sampling scheme identical. The present disclosure includes methods for knowing whether the information content of the desired outcome will remain intact (for example whether it be a fully-reconstructed signal or just a biometric of interest).

These methods can involve computing the uncertainty in signal reconstruction. When the uncertainty is too high, the sampling rate can be increased and/or the transmit power can be increased depending on which one is most likely to increase the information content for the lowest power cost. In at least one example, changing the distribution of sampling intervals can have the largest effect on information content. For example, randomly subsampling sensors 145 is one custom mode for reducing the number of samples. Other sampling techniques may include alternating between sampling rates, using phase-locked loops, and other custom sampling methods. Reducing the number of samples required to reconstruct a signal or measure a biometric reduces the power required by the device 100, thereby increasing the runtime between charges. Since many wearable devices 100 are power limited due to battery size constraints, reducing the power requirements for sensing provides immediate and tangible benefits for end user experience. In at least one example, the sensor or sensors 145 can include a photoplethysmogram (PPG) sensor or sensors. These types of sensors 145 are sensitive to artifacts caused by motion of the body. For example, when a person is walking or engaging in an activity, the sensor or sensors 145 might have artifacts or disturbances caused by activity. The present disclosure implements one or more accelerometers and uses custom sampling interval distributions to subsample the accelerometer values and reconstruct motion artifacts thereby allowing for correction or removal of motion from the sensor data. Other types of sensors 145 as contemplated by the present disclosure include an accelerometer and/or gyroscope, electrodermal activity sensor, and electrical impedance sensor.

When sensor streams are sampled randomly (compared to full resolution sampling), it may not be immediately clear whether the random subsample includes sufficient information to reconstruct the signal of interest, or to predict the biometric of interest. However, by examining the output of one or more of these sensors 145, the sampling rate can be adjusted in real time to ensure that the signals can be reconstructed and the biometrics can still be measured or predicted with high confidence. The problems involved can include: (1) being able to adjust the random sampling rates on the fly for one or more sensors 145 based on the outputs of other sensors 145; (2) being able to estimate signal quality for biometric reconstruction in real time so that random sampling may be adjusted at the hardware level; (3) estimating uncertainty in signal quality as a result of the random sampling so that it may be adjusted on the one or more sensors 145 sampling randomly; and/or (4) reconstruction of the original signal as accurately as possible using the random samples.

In connection with these problems, the present disclosure includes: (1) sampling one or more sensors 145 randomly where the time between samples is customized on the fly using a sampling interval probability distribution; (2) adjusting the sampling distribution based on the values obtained by one or more other sensors 145; (3) estimating quality of the reconstructed signal without fully reconstructing it so that adjustments can be made in real time; (4) reconstructing approximations to the full-resolution signals using the random samples; and/or (5) predicting biometrics that usually require a full-resolution signal with only the random subset.

Conventionally, a process is described to randomly sample a sensor, and then the power spectrum is estimated using Lomb-Scargle. However, the present disclosure describes the processes whereby multiple sensors 145 may be coupled together to adjust the distribution of random samples. Conventional processes also fail to assess the uncertainty in reconstruction for either the entire signal, or the derived biometrics that depend on the signal. Apart from estimating uncertainty in reconstructing the entire signal, conventional processes also do not reconstruct the signal. The present disclosure relies on the multi-modal coupling of random and custom sampling distributions and the corresponding calculation of uncertainty and reconstruction of an approximation to the full-resolution signal using the customized subset of samples.

A random sampling distribution includes an array of integer bin counts, where each bin represents 1 millisecond (ms). Adjusting the number of entries in each bin changes the probability of that interval being chosen at random. For example, the first bin represents an interval of 1 ms. If the sum of all entries in the bin is 1000, and the first bin has 2 entries, then the probability of picking a sampling interval of 1 ms is 2/1000. The second bin would represent the probability of picking an interval of 2 ms, and so on. To pick the random interval, a random integer is picked between 0 and the total sum of all entries across all bins. Then, the bins are cumulatively summed until a bin is reached whose cumulative sum exceeds the random integer picked. When an uncertainty calculation (described below) exceeds the acceptable threshold for the signal or biometric of interest, the distribution is skewed toward shorter intervals by moving bin counts from bins on the right (at higher intervals) to the left. This means that more random samples will be generated (due to shorter intervals between sampling), which allows reconstruction of the original signal with greater certainty. Conversely, if the uncertainty is too low, the distribution may be skewed toward larger intervals, thus saving power.

In real-life use of wearable sensors 145, fit is a critical issue that affects data quality. Unfortunately, a good fit is seldom constant for any one user throughout the day. Instead, the goodness-of-fit changes with temperature, humidity, swelling of the skin, exercise level, swimming/showering, etc. Thus, modifying the sampling distribution must go both ways: (1) toward shorter intervals when uncertainty increases so that reconstruction is still possible; (2) toward longer intervals when the fit is good and uncertainty is low so that power can be saved.

As described above, the modification of the sampling intervals correlates with the uncertainty in the reconstruction/prediction of the signal or biometric quantity of interest. Since reconstructing a full signal is CPU intensive, it cannot conventionally be performed on a microcontroller with limited battery. Many predictions based on random sampling are similarly constrained. Thus, there needs to be a simpler, more energy-efficient way to determine uncertainty in the sampled signal. In as much as good quality signals typically have a well-known frequency distribution (even if that distribution is not stationary), estimating the frequency content for a randomly-sampled signal is a useful place to start.

Sources of uncertainty can include one or more of the following: (1) Multiple LSP compression ratios. (2) Flatness of power around target frequency. For example, if the neighboring frequency's power is more than 3% lower than the target frequency's power, the peak is not flat. (3) Power at harmonic frequencies (integer multiples of the target frequency). For example, if the target frequency is 1.5 Hz, then look at the power at 3 Hz and 4.5 Hz. Uncertainty is the fraction of harmonic power relative to the mean power across several random frequencies. Flatness uncertainty can be used at each harmonic as well. (4) Prior information from the biometric of interest from population statistics. This is the population variance around the target frequency.

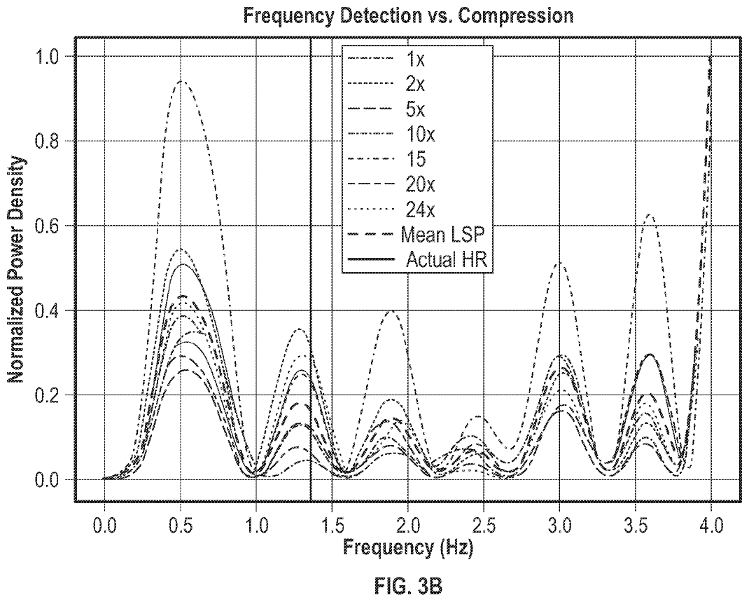

An example of a simple method to compute frequency content for a randomly-sampled signal is the Lomb-Scargle Periodogram (LSP) (see for example FIG. 3A). This method can be used in connection with random sampling of sensor data. The LSP method is able to run on fixed-point microcontrollers. In the present disclosure, the LSP is utilized to estimate uncertainty, for example estimating uncertainty for a multi-sensor/multi-modal distribution of randomly sampled time series data streams. At a high-level, LSP is a combination of Bayesian and Compressed Sensing methods for non-uniformly sampled signals that has common-sense tradeoffs between the upside and downside of each method. Given a set of random samples, the LSP estimates the frequency spectrum. If the number of random samples available exceeds the minimum threshold for producing a reasonable power spectrum, a second level of random samplings (for example resample the random samples again, to produce multiple subsets of random samples) produces multiple estimates for the actual periodogram. Thus, at any given frequency, a distribution of estimates for the power at that frequency can be produced. It is worth noting that the LSP is computed one frequency at a time, so there is no need to compute the entire spectrum.

For each of the sensors 145 being randomly subsampled, we create a-priori a list of frequencies that are “characteristic” for the features of interest. The features of interest for detecting heart rate, for example, can be extracted from a population distribution of heart rates. If the feature of interest is respiration rate, a different distribution is of interest since breathing rates seldom exceed 30 breaths per minute, and usually are closer to 10-20 for most people. With knowledge of these distributions, a set of subsampled LSPs can be created after each epoch and the uncertainty in the frequencies of interest can be computed. Since the LSP is smooth, it is unnecessary to estimate uncertainty at fine granularity. For example, if the biometric of interest is heart rate, it is unnecessary to compute the LSP at 59, 60, 61 BPM etc. Instead, computing every 15 BPM is sufficient to compute the uncertainties. This same method can be applied to each of the multiple sensors 145 being used.

Additionally, the selection of the several potential random intervals can be the one that has low enough total motion in the accelerometer, and/or be based on heuristics derived from any of the other sensors 145. Additionally, instead of correcting only for the motion after the sample has been done, the present method and device 100 can randomly sample for reduced motion at the device 100 and/or sensor (for example in real time).

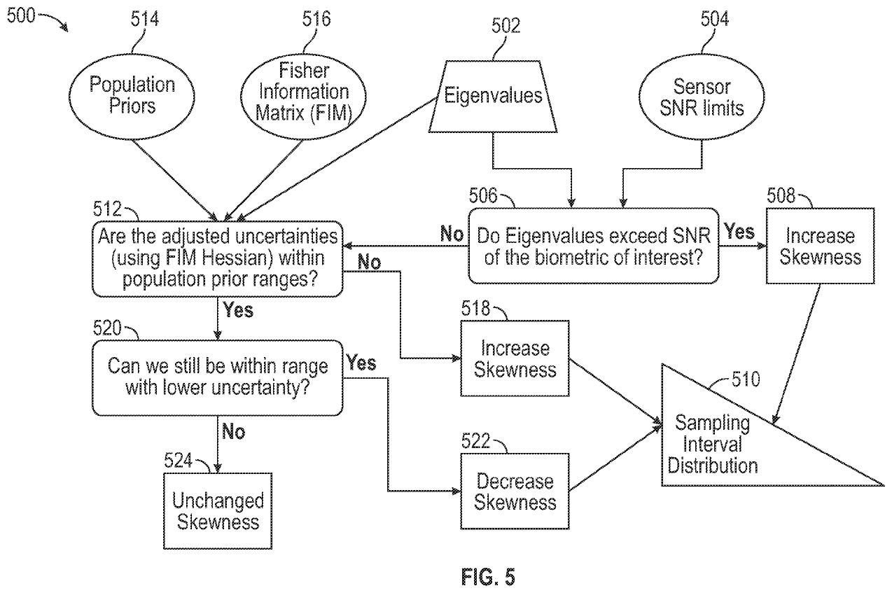

Once uncertainties are available for each of the sensors 145 and quantities of interest, a matrix is constructed for each combination of sensors 145 with entries being the uncertainty of each sensor's random samples. This matrix is used to decide whether the sampling distribution should be skewed toward higher or lower random sampling rates (see for example FIG. 5, described further below). In as much as there are many published ways to use such an uncertainty matrix, we present just two here as representative:
(1) The eigenvalues of the matrix describe the combined uncertainty for each of the sensors 145. Higher eigenvalues mean greater uncertainty. Therefore, the distribution would be skewed to favor shorter sampling intervals. These adjustments can be proportional to the eigenvalues.
(2) The Fisher Information Matrix can be computed using knowledge of the quantities and signals of interest. For example, if blood pressure is predicted using a combination of PPG and EDA sensors 145, the Hessian matrix is computed offline of the features from those signals and how they influence the prediction. The eigenvalues of the Fisher matrix can be stored on the microcontroller and combined with the eigenvalues of the uncertainty matrix for an even better estimate of uncertainty.

The Fisher Information Matrix (FIM) describes the variance in reconstructing the signal as a function of the input variables (random sensor samples). The FIM describes the curvature of the log likelihood graph. As an example, near the maximum likelihood estimate, the Fisher information describes how sharp the maximum is. Higher information means a sharper maximum. Lower information means a flatter maximum. If the maximum is flat, it means that there are many nearby values with a similar likelihood. This is correlated to lower certainty in the final prediction (because there were many other values that were equally likely).

In any case, combining the eigenvalues of the LSP uncertainty matrix yields a quantity that allows modification of the distribution of sampling intervals in real time to provide reasonably high confidence that the biometrics of interest can be predicted, or the signal can be reconstructed well enough using the random samples. Note that in the case of modulating sensor transmit power, the Fisher Information Matrix becomes extremely useful because there is a non-linear relationship between the information content in the signal and the transmit power. This can change from sensor to sensor. Although the uncertainty calculations are still valid and useful, it is less clear how much to increase the transmit power to decrease uncertainty (compared to the sampling interval distributions). Having eigenvalues from the Fisher Information Matrix estimates exactly how much the information content is likely to change as the transmit power varies.

In cases where the biometric of interest is directly related to the frequency content of the full resolution signal, it may be necessary to detect peaks in the LSP. The present disclosure can use all of the power peaks in the LSP, not just the largest one. Additionally, the present disclosure provides for broadening detected peaks using a peak flatness criterion to provide uncertainty in frequency for each detected maximum. When there is insufficient random data, the LSP looks similar to a decaying exponential or may have several regions that are mostly flat. The peaks in these situations may still be detected as peaks based on signal derivatives, but they will not be reflective of the actual frequency content in the signal. Thus, flatness can be estimated using prior knowledge of the biometric quantity of interest (for example characteristic frequency spectra in clean, full resolution PPG data for heart rate). This flatness value changes the uncertainty described above.

Additionally, the present disclosure computes an additional uncertainty based on aliasing at integer frequencies for each peak. For example, for aliasing uncertainty, a probability can be calculated at each possible alias using the population prior information. Valid high-power frequencies should have harmonics at higher frequencies. Thus, the present disclosure also suggests aliased predictions based on significant power around exact integer multiples within the LSP. Similarly, if harmonics are absent when they are expected, this can further increase the uncertainty.

Further still, the present disclosure can form joint probability distributions for all combinations of peaks and aliased suggestions. Then the present disclosure can use weighted combinations based on the integrated power surrounding each prominent peak in the LSP, taking the flatness criterion into account.

The present disclosure may compute the shape and/or width of each LSP peak by fitting a normal or skewed normal distribution and computing residual from the fitted distribution, standard deviation of the fitted distribution, locations of maximum residuals, and/or asymmetry in residuals (for example rise vs. fall). These residuals are used as further sources of uncertainty, depending on the biometric of interest.

When multiple sources of uncertainty are present as described here, the uncertainty must be propagated to create the final uncertainty value for the sensor as a whole. This combined value is the one that should be used in the matrix above before computing eigenvalues. The present disclosure can use one or more uncertainty propagation methods as an ensemble to predict total uncertainty, or uncertainty with respect to specific biometrics and sensors 145. Examples of methods that can be used include Bayesian prior personalized to the user wearing the device 100, Markov-Chain analysis, and particle filtering to predict continuous biometric values. Additionally, selecting the optimal prediction using a directed acyclic graph of predictions with probabilistic weights on the edges and using a standard graph optimization technique including but not limited to shortest path, eigen decomposition of the Laplace matrix, page rank, and the like can be implemented either instead of the above analysis or in addition to the above analysis. Such CPU-intensive algorithms could be run on an additional connected device 100 with greater power availability, and then communicated by a connection 105, for example via Bluetooth, lower power radio communication; and/or ZigBee. Though in certain cases, the microcontroller may have sufficient power to run it local to the sensors 145.

This brings us to the topic of reconstruction. Reconstructing a signal using a subset of random samples is known as compressed sensing. Compressed sensing relies on a quality of the sensing and representation bases known as “incoherence”. As long as the incoherence between these bases is sufficiently high, the signal can be reconstructed with increasing accuracy. Accuracy of reconstruction thus depends on (1) the number of samples; (2) the incoherence of the bases. For most bases, a random sampling of the signal is sufficiently incoherent to allow reconstruction as long as the samples are independent and identically distributed in a Gaussian sense and the representation basis is orthogonal. Additionally, it is a hard requirement in practice that the representation basis be able to sparsely represent the signal. For signals that have limited frequency content, the Fourier basis is sparse. For example, if the signal of interest is a sine wave of 60 kHz, then the information content in the wave is only a single point in the Fourier basis, therefore the signal can be represented sparsely in that basis. At the other extreme, a Heaviside Theta (step) function is not sparse in the Fourier basis, but the function will be sparse if DeHaar wavelets are used. Finding the correct representation basis is thus non-trivial for new signals that are not sparse in any of the well-known mathematical bases.

The present disclosure provides an iterative method for signal reconstruction that is effective for biological signals that are slowly varying, for example, the PPG signal. For many biological/biophysical systems, the quantities of interest vary in ways that mimic combinations of Gaussian or Boltzmann distributions. Using a vanilla Gaussian or Boltzmann distribution, however, does not usually lend itself well to compressed sensing because it lacks compact support. This makes the reconstruction step numerically unstable and difficult to work with (due to infinite integrals). For most compressed-sensing problems, the sparse reconstruction within the representation basis is achieved using L1-regularized optimization such as LASSO, Split-Bregman, or other Bayesian-based methods. For a biophysical signal that meets the criteria described above, a new representation basis can be created by adding an L1 regularization term to the variational principle for Gaussian-like basis functions, which yields solutions with compact support. Linear combinations of these basis functions approximate the eigenvalues and eigenfunctions in a systematically-improvable manner. Thus, if the biophysical signal of interest is approximately sparse in a Gaussian-like basis with compact support, it will be reconstructable within this basis. Since most biological phenomena are restricted in time (for example a single heartbeat does not affect the signal significantly past the next heartbeat, thus it is localized in time), this basis is generally good to use.

Another approach to construct a custom basis uses custom wavelet families. Multiresolution analysis (MRA) is an established method for creating discrete wavelet families that satisfy the admissibility criterion. The iterative approach to create a custom family for this disclosure are: (1) estimate the mother wavelet filter bank by taking a series of low-pass, windowed Fourier filters to estimate the non-stationarity of the low frequency components of the signal; (2) iteratively adjust the filter bank coefficients for the mother wavelet until the desired reconstruction error is low enough for the low frequency components; (3) apply MRA to produce additional wavelets using the scaling functions.

Note that this method assumes an inherent “fractalness” to the form of the signal being reproduced. For biological signals, this is frequently true which is why this methodology is used in the present disclosure.

The steps to reconstruct the signal can include the following:

Step (1) For each random sample from the signal, create a row in the representation matrix by computing the value of each basis function (columns) corresponding to that position (in time) in the ideal signal. For example, with PPG signals, a single PPG waveform will change shape based on the heart rate. Computing the basis functions at various scales and positions along the ideal PPG waveform provides a set of representations at the same point in time that may correlate with the random sample. Each row in the representation is separated from the previous one by a known interval ‘t’. The optimization step below will pick that subset of the basis functions that can best reproduce all random samples in the signal.

Step (2) This will create a large, M×N matrix, where M>>N meaning that there are more basis functions than random samples.

Step (3) Perform an L1 optimization to try and predict the random samples of the signal.

Note that multiple matrices can be constructed at key points along the ideal signal. The final solution is selected as the one with the smallest number of coefficients (i.e., the sparsest signal). Performing this optimization multiple times is CPU intensive. However, the matrices from step (1) above can be tabulated and calculated ahead of time for prototypical signal morphologies.

The quality of the reconstruction is roughly, inversely proportional to the number of coefficients kept in the reconstruction. If it is known that the signal can be represented sparsely in the representation basis, then a non-sparse reconstruction can be the result of: (1) insufficient random samples; and/or (2) bad signal quality.

Note that if the noise in the signal is normally distributed, then it will automatically be excluded by the L1 optimization. Thus, bad signal quality does not mean “normally distributed noise”, but usually represents bad fit or sensor issues. If none of the reconstructions from the steps above are sparse, then it is likely that the signal quality is insufficient. This knowledge is used to tweak the eigenvalue-interval mapping for changing the random sampling distribution in real time.

Predicting a derived biometric quantity, such as heart rate, can follow the same steps. Instead of the basis functions being the compressed Gaussian modes with compact support, feature matrices derived from the ideal signals can be used instead. Note that any of the steps described above for computing uncertainty from randomly sampled values may also be used in the reconstruction or prediction phases. This is especially true for biometrics that are closely related to the frequency content of the full-resolution signal (for example heart rate or respiration rate). In general, periodogram features usually contribute to almost all biometric models in some way. The present disclosure also uses one or more standard linear and/or non-linear heuristic, stochastic, and/or deterministic models to weight contributions from one or more of the above features to predict biometrics, or the uncertainty in predicted biometrics.

Finally, computation of the LSP, derived values, and uncertainties may be carried out on the microcontroller/sensor device 100 or a mobile phone communicating using a connection 105 such as Bluetooth, lower power radio communication, ZigBee, directly over USB, and/or any available wireless or wired communication medium. They may also be carried out on a remote server with values sent to the mobile phone through the internet. When the distribution of sampling intervals needs to be updated, the details may be computed on the phone or in the cloud and sent to the device 100 via a connection 105 for example, via Bluetooth, lower power radio communication; and/or ZigBee, to change the actual sampling rate. Since there is inherent latency in network communication, if the sampling rate must be changed frequently and rapidly, it may be impractical to use cloud-based computing to adjust the interval. In these cases, the proposed method provides solutions that can be implemented in fixed point arithmetic on the sensor device 100 directly to overcome such limitations (or implemented with any similarly-constrained computing methodology).

For further optimization, the individual controller chips of the respective sensors 145 can include analog support for the skewed random sampling distributions, and expose state flags for setting full resolution vs. random sampling, with preset distribution options at increasing sample rates. In this case, the microprocessor can update the distribution state of the sensor controllers each time it collects the FIFO data. For example, start with continuous for the first 10 seconds, then change to sparse data but with a relatively fast sample rate, then reduce rate further.

As an extension to the disclosure, the sensors 145 may alternate between random sampling and full-resolution sampling. For example, if the uncertainty becomes too low, a full-resolution sampling can be enabled temporarily to provide a known, good baseline for subsequent random sampling. In at least one example, the present disclosure implements a technique to use compressed sensing and then implementing standard sensing. This can be implemented based upon set times or alternatively implemented via machine learning characteristics (for example uncertainty thresholds). In the timing example, the device 100 can sample at a high resolution for a few seconds when the algorithm first starts to set a well-known baseline or when it needs to obtain more data to configure one or more of the settings. In other examples, it is possible to use compressed and standard sensing in a varied manner so that one is used and then another is used to alter the sparseness of the sampling based on attributes of the signal or other correlated signals. This allows the system to choose between full sampling (or standard sampling) and random sampling based on the uncertainty in the predictions. In another example, the full resolution signal may use a different sensor configuration that is lower power. For example, in the PPG sensor, red light uses less energy than blue light, so the full resolution sampling may use the red LED while the compressed/random sampling may use any of them. Similarly, each color LED may have its own random sampling interval distribution dictating when samples are taken.

In a similar vein, for impedance spectroscopy, different frequencies can have physiological content that can vary at different rates. Depending on the biometric quantity of interest, the frequencies may be selected using similar random sampling techniques, except with a different distribution per frequency. Thus, the present disclosure is easily applied to multiple, additional contexts.

As a further example toward the general disclosure of adjusting sampling interval distributions based on the desired information content in the signal. If the goal was to reconstruct a high-frequency (400 Hz) PPG signal at reduced power. Start at 50 Hz to get a bead on the signal that includes an estimator to track it. The uncertainty methods disclosed above suffice, especially the methods to propagate uncertainty in peak positions in the periodogram. Next use a phase lock loop set at 400 Hz for a single 32 sample window. This leaves only a short interval (for example <80 ms) to try to center the next PPG transition on. Since it does not require being able to consistently retrieve the data from the sensor, this is still possible. The sensor can be configured to do random/compressed sampling in between. This demonstrates the general principle of modifying sampling interval distributions using prior knowledge of the desired information content in the reconstructed signal.

In connection with the general discussion of lowering power consumption in sensors, high power sensors on wearable devices 100 are often the gyroscope, radios (GPS, WI-FI, BLE), PPG, light spectroscopy (multi wavelength PPG), and/or impedance (AC). Thus, any of these sensors can implement the example discussed here to benefit from lower power consumption.

The present disclosure can implement one or more of the above features. In some examples, all of the above can be combined. The present disclosure provides for an accurate heart rate to be predicted using much lower energy (in some cases 40× lower). Other biometric and full-resolution signals can also be reconstructed using much lower power.

Furthermore, in at least one example, the present disclosure can also be used in predicting respiratory rate. Combining heart rate variability with electrodermal activity (EDA) and further examining the set of randomly subsampled LSPs allows predicting respiratory rate. The present disclosure may also obtain a stress score and combine it with heart rate variability. Additionally, the above techniques can also be used to detect oxygen saturation (SpO2) hydration, blood pressure, blood glucose, tidal volume, and/or electrodermal activity.

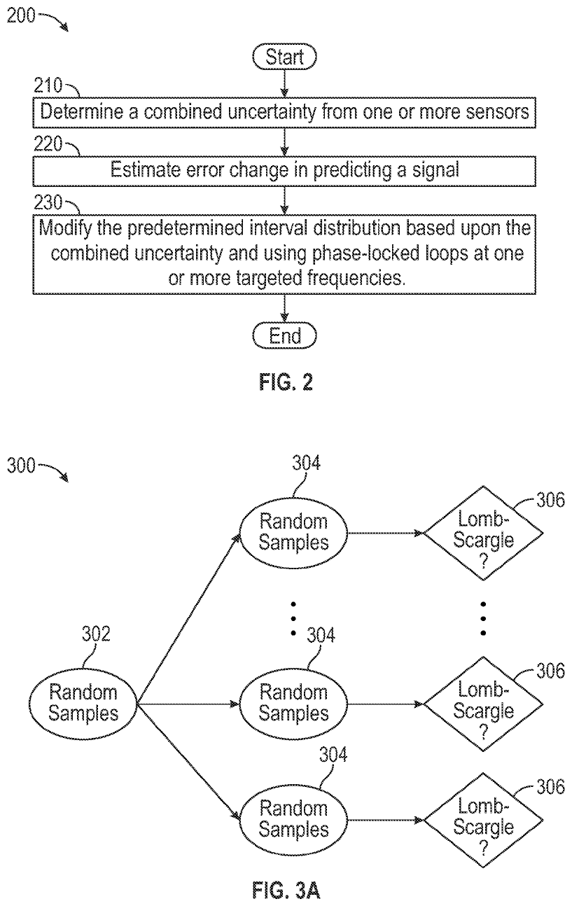

Referring to FIG. 2, a flowchart is presented in accordance with an example embodiment. The method 200 is provided by way of example, as there are a variety of ways to carry out the method. The method 200 described below can be carried out using the configurations illustrated in FIGS. 1 and 3A-5, for example, and various elements of these figures are referenced in explaining example method 200. Each block shown in FIG. 2 represents one or more processes, methods or subroutines, carried out in the example method 200. Furthermore, the illustrated order of blocks is illustrative only and the order of the blocks can change according to the present disclosure. Additional blocks may be added or fewer blocks may be utilized, without departing from this disclosure. The example method 200 can begin at block 210.

At block 210, a combined uncertainty is determined from one or more sensors. A lower resolution can be sampled at a predetermined interval distribution of one or more sensors: accelerometer, electrodermal activity sensor, photoplethysmographic (PPG) sensor; impedance sensor, gyroscopic sensor, and/or a radio sensor.

In at least one example, the determination of the combined uncertainty can be performed on a distinct device from a device that contains the one or more sensors. The distinct device and the device can be electronically coupled. The electronic coupling can be achieved using one or more connections, such as Bluetooth, lower power radio communication, and/or ZigBee. A desired adjustment to the predetermined interval distribution can be transmitted to the device. In at least one example, the distinct device can include a server and/or a cloud computing device.

In at least one example, the determining of the combined uncertainty can be made using one or more of the following methods: multiple, independent random sub-samples to produce multiple Lomb-Scargle Periodograms (LSPs); flatness criteria from peaks of several LSPs; aliasing considerations of harmonics at integer multiples of the one or more targeted frequencies; prior information based on population statistics for a biometric of interest or personalized information from health records of an individual wearing a device; eigenvalues of a combined uncertainty matrix for all of the one or more sensors being sampled; and/or eigenvalues of a Fisher information matrix for the biometric quantity of interest with respect to each of the one or more sensors being sampled.

FIG. 3A illustrates a diagram 300 showing creation of random samples to compute uncertainty from a single sensor. From a starting set of random samples 302, additional random subsets 304 of the original sampling are created. This creates multiple candidates 306 (for example LSP) with different compression ratios. The mean periodogram and its standard deviation provides a base uncertainty measure for a given sensor. FIG. 3B is an example for a PPG sensor of finding the uncertainty (shaded gray area) for multiple frequencies by looking at many compression ratios (many random sub-samplings of an original random sampling).

At block 220, an error change is estimated in predicting a signal.

At block 230, the predetermined interval distribution is modified based upon the combined uncertainty and using phase-locked loops at one or more targeted frequencies, which are adjusted in real-time. In at least one example, the modification of the predetermined interval distribution can implement: a high frequency phase locked loop to capture a dicrotic notch in a PPG signal; a lower frequency sampling post-crest with sufficient resolution to maintain the phase-locked loop; and an even lower frequency sampling in between to ensure locking to morphological features of the PPG signal such as notch and crest. The high frequency can be about twice the lower frequency and the even lower frequency is about a quarter of the lower frequency. For example, the high frequency can be about 400 Hz, the lower frequency can be about 200 Hz, and the even lower frequency can be about 50 Hz.

The sampling of the one or more sensors can be modified, in real time based on the estimated error change, to be one of random, sparse, and/or high resolution.

A custom transmit power modulation can be generated. The custom transmit power modulation can be modified based upon the combined uncertainty and/or estimated error change.

In at least one example, the sampling can be changed between random, phase-locked loop and full-resolution based on the combined uncertainty. The sampling can be a random sampling interval distribution and controlled by a state machine of a controller of the one or more sensors.

In at least one example, the method can further include receiving data from the one or more sensors; receiving data from at least one accelerometer; generating motion artifacts from the received data and the at least one accelerometer; measuring heart rate; modifying, based on the generated motion artifacts, the measured heart rate; creating a set of candidate biometric (for example candidate heart rate) predictions based on Lomb-Scargle Periodogram using non-periodic, randomly, and/or custom sampled data, and also combining one or more steps of: harmonic detection, anti-aliasing, uncertainty propagation and additional Lomb-Scargle subset computations, Markov chain particle-filtering, and/or standard ensemble voting; selecting a best candidate biometric.

In at least one example, data can be received from the at least one or more sensors. Data can be received from at least one accelerometer. Motion artifacts can be generated from the received data and the at least one accelerometer. A full-resolution sensor data can be reconstructed using the random or custom subsample and generated motion artifacts. At least one biometric of interest can be detected using the full-resolution sensor data. In some examples, high frequency impedance measurements can be received at a single frequency within a range of above 0 Hz to 100 kHz using a random or custom subsample from a skewed normal distribution, adjustable in real time using multi-sensor uncertainties; motion artifacts can be generated from the at least one accelerometer; the full-resolution sensor data can be reconstructed using the random or custom subsample of data and the motion artifacts; and the at least one biometric of interest can be predicted.

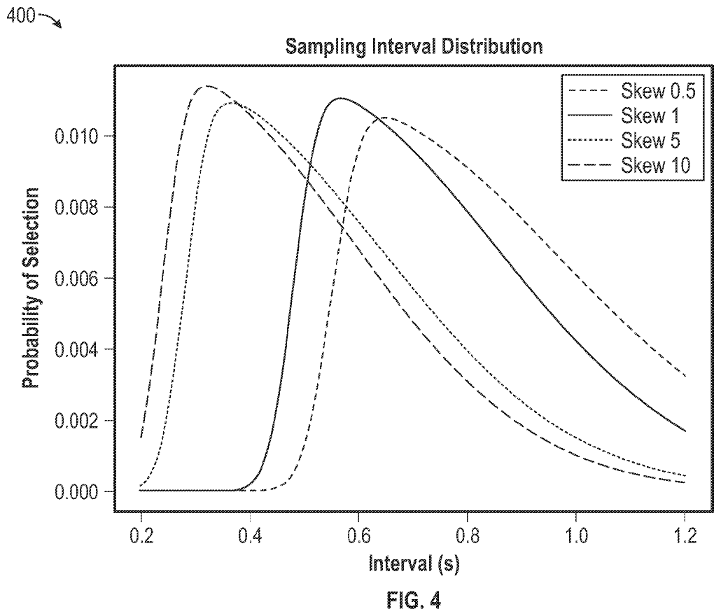

FIG. 4 illustrates a graph 400 showing probability of selection vs. sampling interval distribution. Random sampling interval probability is adjusted using a skew parameter. Increasing the skewness creates intervals that are closer together, meaning that more random samples are created and thereby the uncertainty in the signal reconstruction is lowered. Decreasing the skewness shifts the sampling intervals to larger times between samples, thus increasing the uncertainty in signal reconstruction.

FIG. 5 illustrates a process for adjusting sample intervals using uncertainty matrix eigenvalues and signal-to-noise ratio (SNR) limits of the sensors 145. Eingenvalues 502 and sensor signal-to-noise ratio (SNR) limits 504 are determined. At step 506, based on the eigenvalues 502 and sensor SNR limits 504, it is determined whether eigenvalues 502 exceed SNR 504 of the biometric of interest. If the eigenvalues 502 do exceed SNR 504 of the biometric of interest, the process proceeds to step 506 where skewness is increased to obtain sampling interval distribution 510.

Returning to step 506, if the eigenvalues 502 do not exceed SNR 504 of the biometric of interest, at step 512, it is determined whether the adjusted uncertainties (using Fisher Information Matrix (FIM) 516) are within population prior ranges 514. If the adjusted uncertainties are not within population prior ranges 514, the process proceeds to step 518 where the skewness is increased to obtain sampling interval distribution 510.

Returning to step 512, if the adjusted uncertainties are within population prior ranges 514, the process proceeds to step 520 where it is determined whether it is possible to be within range with lower uncertainty. If yes, the process proceeds to step 522 where the skewness is decreased to obtain sampling interval distribution 510. If no, the process proceeds to step 524 where skewness is unchanged.

Numerous examples are provided herein to enhance understanding of the present disclosure. A specific set of Aspects are provided as follows.

Aspect 1. A method of creating a low-powered approximation of a one or more completed sets of data, comprising: sampling, at a predetermined interval distribution, a lower resolution of one or more of sensors: accelerometer, electrodermal activity sensor, photoplethysmographic (PPG) sensor; impedance sensor, gyroscopic sensor, and/or a radio sensor; determining a combined uncertainty from the one or more sensors; estimating error change in a predicting a signal; modifying the predetermined interval distribution based upon the combined uncertainty and using phase-locked loops at one or more targeted frequencies, which are adjusted in real-time; modifying, in real time based on the estimated error change, the sampling of the one or more sensors to be one of random, sparse, and/or high resolution.

Aspect 2. The method of Aspect 1, further comprising: generating a custom transmit power modulation; modifying, based upon the combined uncertainty and/or estimated error change, the custom transmit power modulation.

Aspect 3. The method of any of Aspects 1 to 2, wherein the determination of the combined uncertainty is performed on a distinct device from a device that contains the one or more sensors, wherein the distinct device and device are electronically coupled; transmitting a desired adjustment to the predetermined interval distribution to the device.

Aspect 4. The method of any of Aspects 1 to 3, wherein the electronic coupling is achieved using one or more of Bluetooth, lower power radio communication; and/or ZigBee.

Aspect 5. The method of any of Aspects 1 to 4, wherein the distinct device is one of a server and/or a cloud computing device.

Aspect 6. The method of any of Aspects 1 to 5, further comprising changing the sampling between random, phase-locked loop, and full-resolution based on the combined uncertainty.

Aspect 7. The method of any of Aspects 1 to 6, wherein the sampling is a random sampling interval distribution and controlled by a state machine of a controller of the one or more sensors.

Aspect 8. The method of any of Aspects 1 to 7, wherein the determining of the combined uncertainty is made using one or more of the following methods: multiple, independent random sub-samples to produce multiple Lomb-Scargle Periodograms (LSPs); flatness criteria from peaks of several LSPs; aliasing considerations of harmonics at integer multiples of the frequencies of interest; prior information based on population statistics for a biometric of interest or personalized information from health records of an individual wearing a device; eigenvalues of a combined uncertainty matrix for all of the one or more sensors being sampled; eigenvalues of a Fisher information matrix for the biometric quantity of interest with respect to each of the one or more sensors being sampled.

Aspect 9. The method of any of Aspects 1 to 8, wherein the modification of the predetermined interval distribution implements: a high frequency phase locked loop to capture a dicrotic notch in a PPG signal; a lower frequency sampling post-crest with sufficient resolution to maintain the phase-locked loop; and an even lower frequency sampling in between to ensure locking to morphological features of the PPG signal such as notch and crest.

Aspect 10. The method of any of Aspects 1 to 9, wherein the high frequency is about twice the lower frequency and the even lower frequency is about a quarter of the lower frequency.

Aspect 11. The method of any of Aspects 1 to 10, wherein the high frequency is about 400 Hz, the lower frequency is about 200 Hz, and the even lower frequency is about 50 Hz.

Aspect 12. The method of any of Aspects 1 to 11, further comprising: receiving data from the one or more sensors; receiving data from at least one accelerometer; generating motion artifacts from the received data and the at least one accelerometer; measuring heart rate; modifying, based on the generated motion artifacts, the measured heart rate; creating a set of candidate biometric predictions based on Lomb-Scargle Periodogram using non-periodic, randomly, and/or custom sampled data and also combining one or more steps of: harmonic detection, anti-aliasing, uncertainty propagation and additional Lomb-Scargle subset computations, Markov chain particle-filtering, and standard ensemble voting; selecting a best candidate hear rate.

Aspect 13. The method of any of Aspects 1 to 12, further comprising: receiving data, from the at least one or more sensors; receiving data from at least one accelerometer; generating motion artifacts from the received data and the at least one accelerometer; reconstructing a full-resolution sensor data using the random or custom subsample and generated motion artifacts; detecting at least one biometric of interest using the full-resolution sensor data.

Aspect 14. The method of any of Aspects 1 to 13, further comprising: receiving high frequency impedance measurements at a single frequency within a range of above 0 Hz to 100 kHz using a random or custom subsample from a skewed normal distribution, adjustable in real time using multi-sensor uncertainties; generating motion artifacts from the at least one accelerometer; reconstructing the full-resolution sensor data using the random or custom subsample of data and the motion artifacts; predicting the at least one biometric of interest.

Aspect 15. A device includes a storage (implemented in circuitry) configured to store instructions and a processor. The processor configured to execute the instructions that cause the processor to: sampling, at a predetermined interval distribution, a lower resolution of one or more of sensors: accelerometer, electrodermal activity sensor, photoplethysmographic (PPG) sensor; impedance sensor, gyroscopic sensor, and/or a radio sensor; determine a combined uncertainty from the one or more sensors; estimate error change in a predicting a signal; modify the predetermined interval distribution based upon the combined uncertainty and using phase-locked loops at one or more targeted frequencies, which are adjusted in real-time; modifying, in real time based on the estimated error change, the sampling of the one or more sensors to be one of random, sparse, and/or high resolution.

Aspect 16. The device of Aspect 15, wherein the processor is configured to execute the instructions that cause the processor to: generate a custom transmit power modulation; modifying, based upon the combined uncertainty and/or estimated error change, the custom transmit power modulation.

Aspect 17. The device of any of Aspects 15 to 16, wherein the determination of the combined uncertainty is performed on a distinct device from a device that contains the one or more sensors and the distinct device and device are electronically coupled; transmit a desired adjustment to the predetermined interval distribution to the device.

Aspect 18. The device of any of Aspects 15 to 17, wherein the electronic coupling is achieved using one or more of Bluetooth, lower power radio communication; and/or ZigBee.

Aspect 19. The device of any of Aspects 15 to 18, wherein the distinct device is one of a server and/or a cloud computing device.

Aspect 20. The device of any of Aspects 15 to 19, wherein the processor is configured to execute the instructions that cause the processor to: change the sampling between random, phase-locked loop, and full-resolution based on the combined uncertainty.

Aspect 21. The device of any of Aspects 15 to 20, wherein the sampling is a random sampling interval distribution and controlled by a state machine of a controller of the one or more sensors.

Aspect 22. The device of any of Aspects 15 to 21, wherein the determining of the combined uncertainty is made using one or more of the following methods: multiple, independent random sub-samples to produce multiple Lomb-Scargle Periodograms (LSPs); flatness criteria from peaks of several LSPs; aliased considerations of harmonics at integer multiples of the frequencies of interest; prior information based on population statistics for a biometric of interest or personalized information from health records of an individual wearing a device; eigenvalues of a combined uncertainty matrix for all of the one or more sensors being sampled; eigenvalues of a Fisher information matrix for the biometric quantity of interest with respect to each of the one or more sensors being sampled.

Aspect 23. The device of any of Aspects 15 to 22, wherein the modification of the predetermined interval distribution implements: a high frequency phase locked loop to capture a dicrotic notch in a PPG signal; a lower frequency sampling post-crest with sufficient resolution to maintain the phase-locked loop; and even lower frequency sampling in between to ensure locking to morphological features of the PPG signal such as notch and crest.

Aspect 24. The device of any of Aspects 15 to 23, wherein the high frequency is about twice the lower frequency and the even lower frequency is about a quarter of the lower frequency.

Aspect 25. The device of any of Aspects 15 to 24, wherein the high frequency is about 400 Hz, the lower frequency is about 200 Hz, and the even lower frequency is about 50 Hz.

Aspect 26. The device of any of Aspects 15 to 25, wherein the processor is configured to execute the instructions and cause the processor to: receive data from the one or more sensors; receive data from at least one accelerometer; generate motion artifacts from the received data and the at least one accelerometer; measuring heart rate; modifying, based on the generated motion artifacts, the measured heart rate; create a set of candidate biometric predictions based on Lomb-Scargle Periodogram using non-periodic, randomly, and/or custom sampled data and also combining one or more steps of: harmonic detection, anti-aliasing, uncertainty propagation and additional Lomb-Scargle subset computations, Markov chain particle-filtering, and/or standard ensemble voting; select a best candidate hear rate.

Aspect 27. The device of any of Aspects 15 to 26, wherein the processor is configured to execute the instructions and cause the processor to: receive data, from the at least one or more sensors; receive data from at least one accelerometer; generate motion artifacts from the received data and the at least one accelerometer; reconstruct a full-resolution sensor data using the random or custom subsample and generated motion artifacts; detect at least one biometric of interest using the full-resolution sensor data.

Aspect 28. The device of any of Aspects 15 to 27, wherein the processor is configured to execute the instructions and cause the processor to: receive high frequency impedance measurements at a single frequency within a range of above 0 Hz to 100 kHz using a random or custom subsample from a skewed normal distribution, adjustable in real time using multi-sensor uncertainties; generate motion artifacts from the at least one accelerometer; reconstruct the full-resolution sensor data using the random or custom subsample of data and the motion artifacts; predict the at least one biometric of interest.

What is claimed is:

1. A method of creating a low-powered approximation of one or more completed sets of data, comprising:
sampling, at a predetermined interval distribution, a lower resolution of one or more of sensors: accelerometer, electrodermal activity sensor, photoplethysmographic (PPG) sensor; impedance sensor, gyroscopic sensor, and/or a radio sensor;
determining a combined uncertainty from the one or more sensors;
estimating error change in a predicting a signal;
modifying the predetermined interval distribution based upon the combined uncertainty and using phase-locked loops at one or more targeted frequencies, which are adjusted in real-time;
modifying, in real time based on the estimated error change, the sampling of the one or more sensors to be one of random, sparse, and/or high resolution.

2. The method of claim 1, further comprising:
generating a custom transmit power modulation;
modifying, based upon the combined uncertainty and/or estimated error change, the custom transmit power modulation.

3. The method of claim 1, wherein the determination of the combined uncertainty is performed on a distinct device from a device that contains the one or more sensors, wherein the distinct device and the device are electronically coupled; transmitting a desired adjustment to the predetermined interval distribution to the device.

4. The method of claim 3, wherein the electronic coupling is achieved using one or more of Bluetooth, lower power radio communication; and/or ZigBee.

5. The method of claim 3, wherein the distinct device is one of a server and/or a cloud computing device.

6. The method of claim 1, further comprising changing the sampling between random, phase-locked loop, and full-resolution based on the combined uncertainty.

7. The method of claim 6, wherein the sampling is a random sampling interval distribution and controlled by a state machine of a controller of the one or more sensors.

8. The method of claim 1, wherein the determining of the combined uncertainty is made using one or more of the following methods:
multiple, independent random sub-samples to produce multiple Lomb-Scargle Periodograms (LSPs);
flatness criteria from peaks of several LSPs;
aliasing considerations of harmonics at integer multiples of the one or more targeted frequencies;
prior information based on population statistics for a biometric of interest or personalized information from health records of an individual wearing a device;
eigenvalues of a combined uncertainty matrix for all of the one or more sensors being sampled; and/or
eigenvalues of a Fisher information matrix for the biometric quantity of interest with respect to each of the one or more sensors being sampled.

9. The method of claim 1, wherein the modification of the predetermined interval distribution implements:
a high frequency phase locked loop to capture a dicrotic notch in a PPG signal;
a lower frequency sampling post-crest with sufficient resolution to maintain the phase-locked loop; and
an even lower frequency sampling in between to ensure locking to morphological features of the PPG signal such as notch and crest.

10. The method of claim 9, wherein the high frequency is about twice the lower frequency and the even lower frequency is about a quarter of the lower frequency.

11. The method of claim 10, wherein the high frequency is about 400 Hz, the lower frequency is about 200 Hz, and the even lower frequency is about 50 Hz.

12. The method of claim 1, further comprising:
receiving data from the one or more sensors;
receiving data from at least one accelerometer;
generating motion artifacts from the received data and the at least one accelerometer;
measuring heart rate;
modifying, based on the generated motion artifacts, the measured heart rate;
creating a set of candidate biometric predictions based on Lomb-Scargle Periodogram using non-periodic, randomly, and/or custom sampled data, and also combining one or more steps of: harmonic detection, anti-aliasing, uncertainty propagation and additional Lomb-Scargle subset computations, Markov chain particle-filtering, and/or standard ensemble voting;
selecting a best candidate biometric.

13. The method of claim 1, further comprising:
receiving data, from the at least one or more sensors;
receiving data from at least one accelerometer;
generating motion artifacts from the received data and the at least one accelerometer;
reconstructing a full-resolution sensor data using the random or custom subsample and generated motion artifacts;
detecting at least one biometric of interest using the full-resolution sensor data.

14. The method of claim 13, further comprising:
receiving high frequency impedance measurements at a single frequency within a range of above 0 Hz to 100 kHz using a random or custom subsample from a skewed normal distribution, adjustable in real time using multi-sensor uncertainties;
generating motion artifacts from the at least one accelerometer;
reconstructing the full-resolution sensor data using the random or custom subsample of data and the motion artifacts;
predicting the at least one biometric of interest.

15. A device comprising:
a storage configured to store instructions; and
a processor configured to execute the instructions that cause the processor to:
sample, at a predetermined interval distribution, a lower resolution of one or more of sensors: accelerometer, electrodermal activity sensor, photoplethysmographic (PPG) sensor; impedance sensor, gyroscopic sensor, and/or a radio sensor;
determine a combined uncertainty from the one or more sensors;
estimate error change in a predicting a signal;
modify the predetermined interval distribution based upon the combined uncertainty and using phase-locked loops at one or more targeted frequencies, which are adjusted in real-time;
modify, in real time based on the estimated error change, the sampling of the one or more sensors to be one of random, sparse, and/or high resolution.

16. The device of claim 15, wherein the processor is configured to execute the instructions that cause the processor to:
generate a custom transmit power modulation;
modify, based upon the combined uncertainty and/or estimated error change, the custom transmit power modulation.

17. The device of claim 15, wherein the determination of the combined uncertainty is performed on a distinct device from a device that contains the one or more sensors, wherein the distinct device and device are electronically coupled; transmit a desired adjustment to the predetermined interval distribution to the device.

18. The device of claim 17, wherein the electronic coupling is achieved using one or more of Bluetooth, lower power radio communication; and/or ZigBee.

19. The device of claim 17, wherein the distinct device is one of a server and/or a cloud computing device.

20. The device of claim 15, wherein the processor is configured to execute the instructions that cause the processor to: change the sampling between random, phase-locked loop, and full-resolution based on the combined uncertainty.

21. The device of claim 20, wherein the sampling is a random sampling interval distribution and controlled by a state machine of a controller of the one or more sensors.

22. The device of claim 15, wherein the determining of the combined uncertainty is made using one or more of the following methods:
multiple, independent random sub-samples to produce multiple Lomb-Scargle Periodograms (LSPs);
flatness criteria from peaks of several LSPs;
aliased considerations of harmonics at integer multiples of the frequencies of interest;
prior information based on population statistics for a biometric of interest or personalized information from health records of an individual wearing a device;
eigenvalues of a combined uncertainty matrix for all of the one or more sensors being sampled; and/or
eigenvalues of a Fisher information matrix for the biometric quantity of interest with respect to each of the one or more sensors being sampled.

23. The device of claim 15, wherein the modification of the predetermined interval distribution implements:
a high frequency phase locked loop to capture a dicrotic notch in a PPG signal;
a lower frequency sampling post-crest with sufficient resolution to maintain the phase-locked loop; and
an even lower frequency sampling in between to ensure locking to morphological features of the PPG signal such as notch and crest.

24. The device of claim 23, wherein the high frequency is about twice the lower frequency and the even lower frequency is about a quarter of the lower frequency.

25. The device of claim 24, wherein the high frequency is about 400 Hz, the lower frequency is about 200 Hz, and the even lower frequency is about 50 Hz.

26. The device of claim 15, wherein the processor is configured to execute the instructions that cause the processor to:
receive data from the one or more sensors;
receive data from at least one accelerometer;
generate motion artifacts from the received data and the at least one accelerometer; measuring heart rate;
modify, based on the generated motion artifacts, the measured heart rate;
create a set of candidate biometric predictions based on Lomb-Scargle Periodogram using non-periodic, randomly, and/custom sampled data and also combining one or more steps of: harmonic detection, anti-aliasing, uncertainty propagation and additional Lomb-Scargle subset computations, Markov chain particle-filtering, and/or standard ensemble voting;
select a best candidate hear rate.

27. The device of claim 15, wherein the processor is configured to execute the instructions and cause the processor to:
receive data, from the at least one or more sensors;
receive data from at least one accelerometer;
generate motion artifacts from the received data and the at least one accelerometer;
reconstruct a full-resolution sensor data using the random or custom subsample and generated motion artifacts;
detect at least one biometric of interest using the full-resolution sensor data.

28. The device of claim 27, wherein the processor is configured to execute the instructions and cause the processor to:
receive high frequency impedance measurements at a single frequency within a range of above 0 Hz to 100 kHz using a random or custom subsample from a skewed normal distribution, adjustable in real time using multi-sensor uncertainties;
generate motion artifacts from the at least one accelerometer;
reconstruct the full-resolution sensor data using the random or custom subsample of data and the motion artifacts;
predict the at least one biometric of interest.
